(19) United States
(12) Patent Application Publication
(10) Pub. No.: US 2019/0192268 A1
(43) Pub. Date: Jun. 27, 2019

(54) ADJUSTABLE DENTAL CASSETTE SYSTEM

(71) Applicant: MARY FADHL, DANVILLE, CA (US)

(72) Inventor: MARY FADHL, DANVILLE, CA (US)

(21) Appl. No.: 15/926,543

(22) Filed: Mar. 20, 2018

Related U.S. Application Data
(60) Provisional application No. 62/508,387, filed on May 18, 2017.

(51) Int. Cl. A61C 19/00; A61L 2/025; A61L 2/07

(52) U.S. Cl. CPC A61C 19/002; A61C 19/02; A61L 2/07; A61L 2/025

(57) ABSTRACT

A novel dental cassette featuring a ‘pop-in’ securing gripper-rails system to clamp odd-shaped dental instruments, while providing adjustable canting, gripping, and drainage capacity through its materials, design and engineering. The silicon gripping posts and feet, and the shovel-and-groove clamps, and the plurality of hex [preferred] holes, provide superior anchorage for dental instruments through autoclave and sterilization processes.

### FIELD OF THE DISCLOSURE

The disclosure relates to the field of dental-instrument cassettes engineered for insertion in ultrasonic and autoclave machines, said cassettes possessing capacity to safely grip, dry and sterilize dental instruments of multiple shapes and sizes.

### BACKGROUND

This invention relates to a universal adjustable dental cassette which grips, stores, displays, holds [for drying] and secures a wide range of dental instruments during the various phases of instrument sanitation while preventing instrument damage, while facilitating superior liquid drainage, while protecting dental hygienists from being poked by loose or protruding instruments.

Dental instrument sterilization cassettes hold soiled dental instruments during sterilization processes. Contaminated instruments are stored in cassettes, which are then placed in autoclave and other steam-cleaning sterilization machines, as well as ultrasonic and other solution-and-agitation cleaning machines. These cassettes customarily come in “hygiene” size and “diagnostic” size, infra.

Sterilization cassettes customarily have apertures for cleaning-vapor or solutions to pass through and agitate the instruments. Cassettes in ultrasonic machines must endure high frequency sound waves' resultant cavitation bubbles, which release contaminants that are stuck to the instruments (dental debris). The holes also provide for future drainage. The instant invention is an adjustable cassette-and-gripper system specifically tailored for all sterilization processes, usually consisting of holding/solution, ultrasonic and sterilization processes. The instant cassette is preferably embodied in two-sizes of the same adjustable cassette which can secure almost any dental instrument (infra) through the autoclave, sterilization, instrument washing, and ultrasonic (enzyme solution) cleaning processes.

Preventing instrument jostling within each cassette is paramount, as instruments can scratch and break within the cassette. In addition, housing and anchoring multi-sized and multi-shaped instruments has heretofore remained an enigma in the industry, as the industry still uses shallow, substantially-fixed, “metal rack style” cassettes which cannot adjust to solve the above issues, and which commonly allow protruding, sharp instruments to stick out of the cassettes and poke dental workers.

No dental cassette has heretofore been invented which addresses all these sterilization and drainage needs. Present cassettes are either too shallow to house larger dental instruments like mouth mirrors and hand-pieces, or their gripping rails/ribs are uniform and parallel, and thus cannot accommodate differently-sized instruments. Furthermore, many cassettes offer polymer bases, which trap debris during sterilization (allowing ongoing contamination) and which provide insufficient drainage (pooling).

In sum, current raw-boxy steel dental cassettes with fixed internal steel internal ribs offer insufficient sterilization and safety. Current polymer cassettes offer slightly better safety, but they also lack in hygiene and stability. An intelligent “universal adjustable” cassette is needed.

### SUMMARY

A novel dental cassette featuring a ‘pop-in’ securing gripper-rails system to clamp odd-shaped dental instruments, while providing adjustable canting, gripping, and drainage capacity through its materials, design and engineering. The silicon gripping posts and feet, and the shovel-and-groove clamps, and the plurality of hex [preferred] holes, provide superior anchorage for dental instruments through autoclave and sterilization processes.

### BRIEF DESCRIPTION OF THE DRAWINGS

The drawings described herein are for illustrative purposes only and illustrate only selected embodiments of the present invention. The enclosed drawings are not intended to limit the scope of the present disclosure.

FIG. 1 is a perspective view of the [in this embodiment] stainless steel tray component of the dental cassette, showing the ‘fold up stamping’ method of manufacture and hexagonal aperture design, herein shown with a double-grooved rail along the tray's top walls, and herein showing two sets of pliable gripping strips/posts.

FIG. 2 is a worm's-eye, exploded view of the tray with grippers inserted in double-row perpendicular transverse fashion and [herein embodiment] equal size feet/stoppers (un-canted).

FIG. 3 is a perspective, worm's-eye view of the tray showing tray depth to allow secure gripping of odd shaped items. Anti-slip feet are herein shown, plugged through the hex-drainage apertures.

FIG. 4 is a perspective aerial view of the tray with a single gripping strip/post holding a single dental instrument (probe), showing secure “shovel top” gripping posts, securing the dental instrument through the rigor of autoclave processes. Adjustability (movability) of said plug/strips is shown, as additional apertures are available to allow dentists to “re-anchor” said probe in the position they want.

FIG. 5 is an aerial side view of the tray with lid cover, as it fits in sterilization machines, showing the gripping strips plugged into the drainage apertures, and showing the unit's canting from differently-sized plug-feet (for drying, to prevent pooling).

FIG. 6 is a worm's-eye view of the tray, featuring parallel grippers/transversal of the dental instruments) and differently-sized feet for canting.

FIG. 7 is a perspective, aerial view of the tray with [herein] same-sized pliable gripper strips (posts) holding multi-shaped dental instruments with its “shovel plug and groove” gripper strip/post tops, said strips anchored (plugged) through the tray's hexagonal drainage apertures.

Optional “grass-like” “bristly” Silicone Clamping-Sheet Insert not herein shown (a pliable pad/sheet that lays atop said dental instruments to further secure them during sterilization). This feature is essentially a rubber sheet inserted between the instruments and the lid.

Optional Convex lid not herein shown (for superior drainage).

Optional Concave tray base not herein shown (for superior drainage).

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an article “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)−(a second number),” this means a range whose limit is the second number. For example, 25 to 100 mm means a range whose lower limit is 25 mm and upper limit is 100 mm.

Aspects of the disclosed invention may be embodied as a system, method or process, or computer program product. Accordingly, aspects of the disclosed invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the disclosed invention may take the form of a computer program product embodied in one or more computer readable media having computer readable program code embodied thereon.

Any element in a claim that does not explicitly state “means for” performing a specified function, or “step for” performing a specific function is not to be interpreted as a “means” or “step” clause as specified in 35. U.S.C. § 112 ¶6. Specifically, the use of “step of” in the claims herein is not intended to invoke the provisions of U.S.C. § 112 ¶6.

As discussed infra, the instant invention secures the following instruments [and more] in a safe, hygienic secure manner preferred by key opinion leaders: probes, retractors, mouth mirrors, dental pluggers and condensers, dental burnishers, prosthodontics instruments, excavators, torque wrenches, chisels, forceps, pliers, and syringes and needle-recappers, ADD scalers and curvettes, and needed accessories.

### DETAILED DESCRIPTION OF THE FIGURES

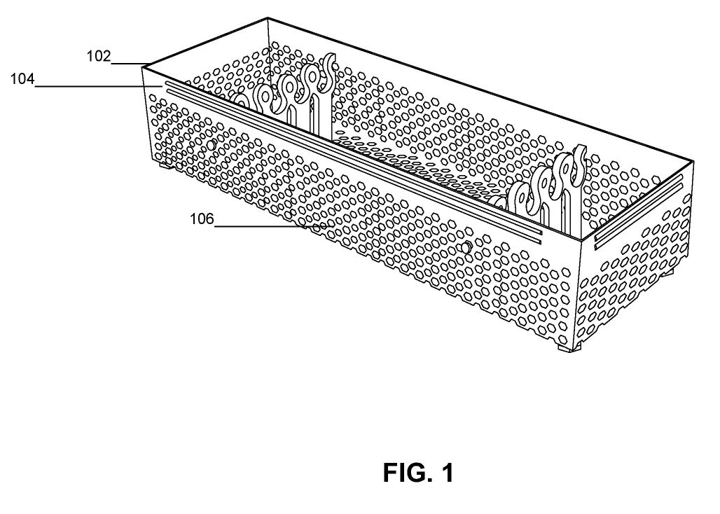

FIG. 1 shows an embodiment of the invention, featuring the “fold up” stamped manufacture of the base/tray. The cassette fits inside most autoclaves, including Midmark™ and Ritter™ autoclaves, Tuttnauer and Scican Statim and Hydrim autoclave machines, sterilizers, and more. The invention also fits inside standard baskets for ultrasonic cleaners. The preferred cassettes are the “5 instrument” embodiment at 7½″×2½″×1.5″ (last dimension=height/depth). Another embodiment is for the “medium 8 instrument” cassette at 7½″×4½″×1.5″ (last dimension=height/depth). Other embodiments come in 8″×4.5″×2″, 7″×3″×2,″ and other dimension cassettes are available.

Reference number 102 is the tray, 104 is the (optional but preferred) grooved rail to engage the notch-ridge along the inside of the lid-cover (not herein shown); 106 drainage apertures throughout, prevent pooling (preferred embodiment hex hole shape has numerous benefits, especially, herein, they hold the pliable (usually silicon) clamping strips 204 [also called posts 402, depending on shape of dental instrument held and length and positioning of strip], to prevent the strips/posts 204 from rotating in place (which would allow the dental instruments to jostle, come loose during sterilization, and accidentally poke dental workers). The (optional but preferred) hex holes 106 therefore aid drainage, and also provide proper anchors for the strip 204 plugs 502 to securely (removably) fasten in place. This hex style prevents accidental disengagement or rotation (which would create a mess-o′-dental-instruments inside the cassette, post-sterilization).

The [preferred] silicone gripper and rail system reduces instrument contact, allowing for cleaning solutions to agitate-away more debris. [One embodiment] Stainless steel housing construction in the tray/base protects against corrosion. Apertured-edges allow for superior drainage and drying. The [preferred] hexagonally shaped apertures offers multiple advantages, discussed infra and supra.

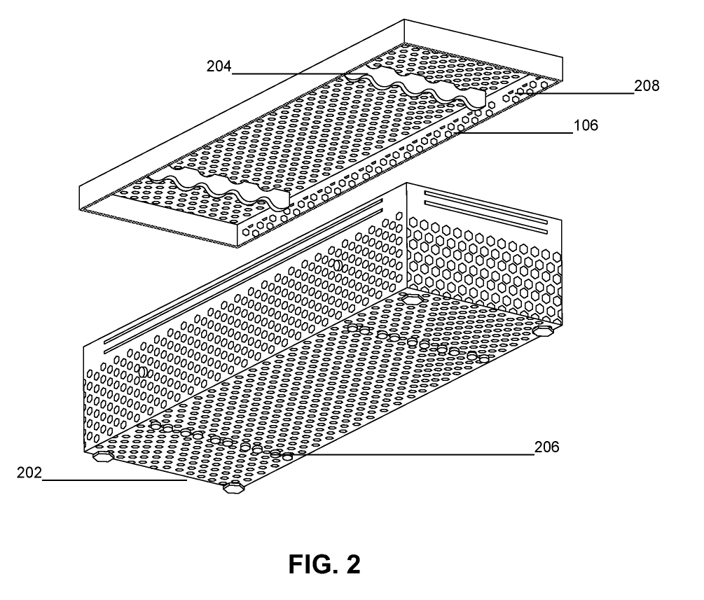

FIG. 2 shows base 202, which may alternatively (not herein shown) be convex (middle up) to prevent pooling, the pliable gripping strip 204, herein taking the form of a securing strip affixed (plugged) to the top via aperture plugs (helps to secure the instruments below during the bumpy-and-steamy autoclave-&-sterilization processes), the lid's notch-ridge 208, while optional, helps secure the lid, the apertures 106 are shown throughout the lid. The lid 210 should, if [preferred] silicon-pliable material, securely fit over tray 102 ridged rails 104 to hold the unit together for autoclave, sterilization, and storage.

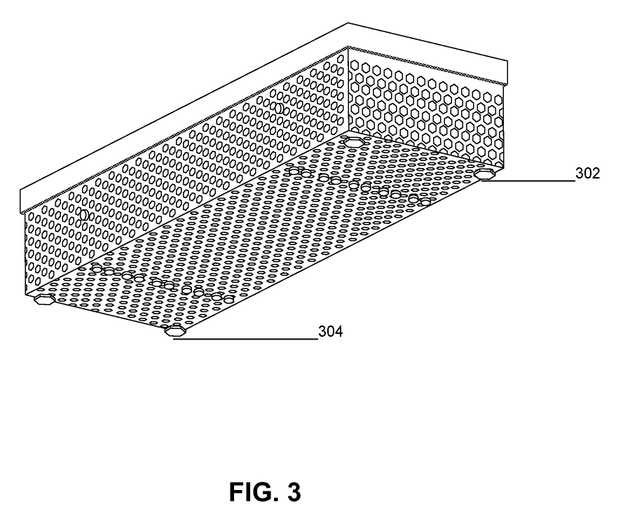

FIG. 3 features the anti-slip feet 302/304, being the same size in the embodiment shown in FIG. 3, but often differently-sized to create base 202 canting in other embodiments (infra). Feet 302 plug through hex apertures 106 as other gripper plugs, and are preferably rubber or silicon or appropriate similar compound thereof (ideally the same material as all the other the strips 204 and posts 402) (Reference numbers 204 and 402 used interchangeably as/where appropriate, as the interchangeability of plug-strip-posts 204/402 (and their alternate sizing) is a key component of the instant invention). All 204/402 strip/posts feature the same “plug style” removable fasteners 502 and (when posts to grab and secure instruments) the shovel-and-groove style clamp tops 404.

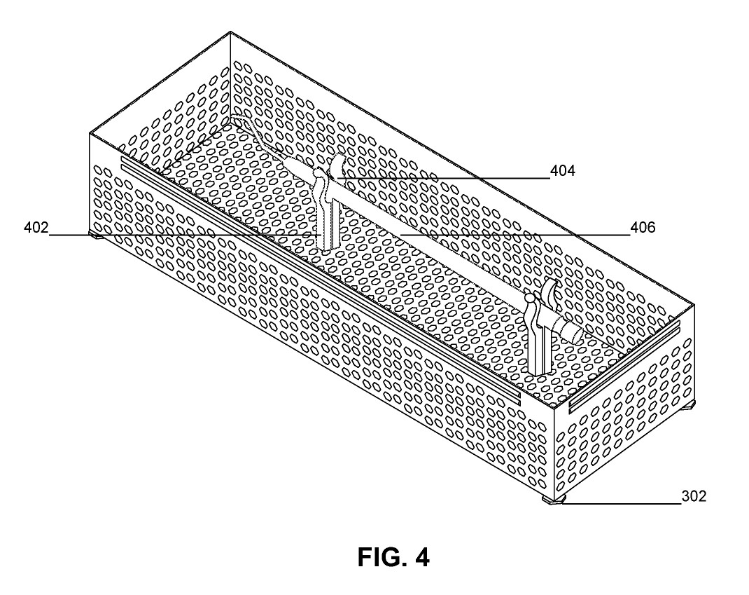

FIG. 4 herein shows differently sized anti-slip feet 302 and 304, respectively, preferred canting not herein shown, the ‘shovel and groove’ shaped clamp-top 404 to the gripping strip/post 402, the clamped dental instrument 406 (herein a “pointy” probe, showing the need for stable clamps).

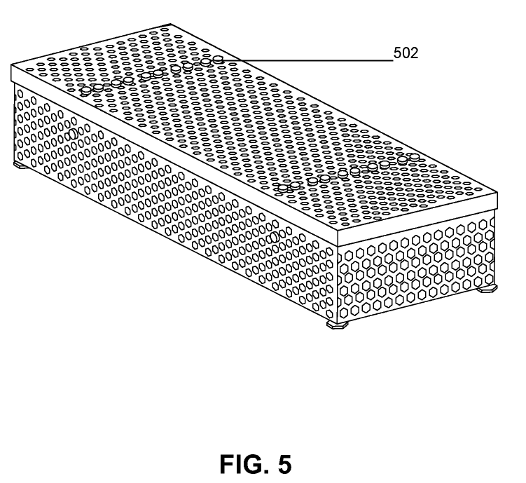

FIG. 5 shows the cassette assembled (lid-on) for sterilization and storage, herein showing the gripping strip 502 plugged through the hex holes 106.

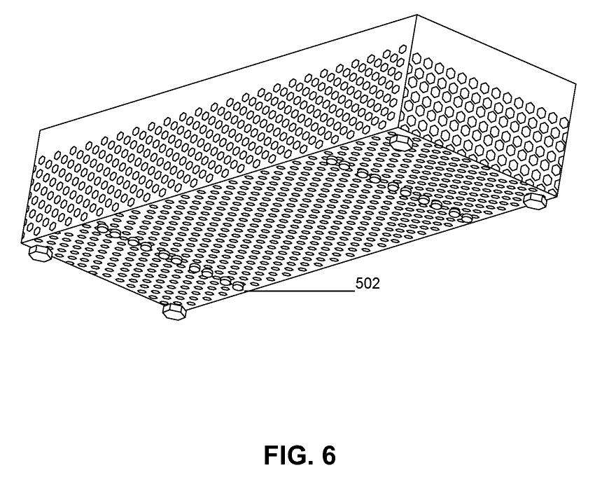

FIG. 6 shows the main tray with gripping strips/posts 402 plugged/secured through the hex base holes 106, herein two strips positioned in parallel to hold elongated dental instruments. As with all the invention's pliable strips/posts 402, different dental instruments may receive different strip/post 402 sizes and configurations, therein making the dentist's “presentation/diagnostic” stage, and latter-sterilization/hygiene processes, easier and “tailor-made” for the instruments s/he is using. Said strips/posts 402 may also be color-coordinated by instrument-type or by patient, or other logical indexing means.

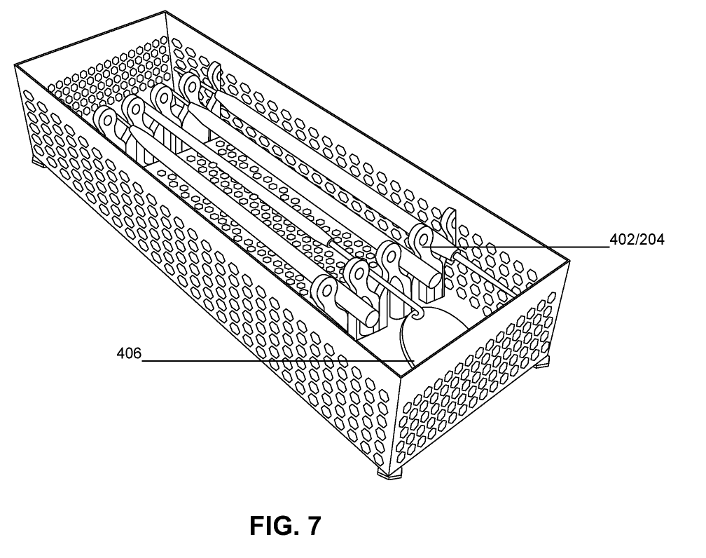

FIG. 7 shows multiple probes 406 and a mirror 406 (all dental instruments 406 for reference purposes) securely held by gripping posts/strips 402/204 (here said strips are arranged in parallel). Note the tray is sufficiently deep to hold canted-mirrors 406, which the gripper posts 402 accommodate due to their “pop-in/pop-out” versatility (allowing various posts 402 to be different heights when needed, such as for odd-shaped instruments like mirrors [unlike probes, which are fairly common in size and shape]).

### PREFERRED EMBODIMENT OF THE INSTANT INVENTION

Cassette feet are silicone-rubber [either or compound or mixture] stoppers 302/304 feet for support, improving drainage and preventing slippage. Small and large feet are optionally positioned on the distal and proximal ends, effectively creating a drainage chute [canting of base 202], as base is then a canted cassette floor for superior drainage. Adjustable color coded “Snap-in/Plug-in” support retainers (“Grippers”) 204/402 to anchor and secure [usually] ⅜″ and thinner probes 406; also for bigger mirrors 406; adjustable walls within the cassette (formed by popping-in-additional-strips 204, not herein shown), also anchored with the hex plugs.

Hexagonal drainage apertures 106 allow proper spillage, prevents pooling and allows instrument debris to flow out of the cassette. Other hole shapes are appropriate for the invention, so long as their width and geometry allows for the secure “pop-in-pop-out” method of securing rails/grippers 204/402. The size, arrangement and shape of [preferred hex] holes (drainage apertures) 106 prevents instruments from sticking/poking out of the cassette and therefore from poking the dental hygienists and other workers.

Stainless steel [preferred] housing 102 with silicone (or similar semi-pliable) (antibiotic-treated) lid 210; adjustable (including diagonal [alternative embodiment, but “easily adjustable by dentist upon arrival]) gripping rail 204/402 design, adjustable for ‘odd shaped’ instruments 406.

[Preferred] Silicone lid cover 210 (rather than steel or metal) (BPA Free), with Triclosan-style (e.g. Microban™) antibiotic added to the lid material (rather than mere stainless steel). The lid 210 pops on and off like Tupperware™ (or similar groove lip and ridge system). The lid also optionally has at least one corner tab for easy-opening (grabbing) (not herein shown).

Lid 210 and Cassette Tray/Base 202 may also have convex planes, to further improve drainage and prevent pooling;

Hygiene Size and Diagnostic Sizes available (5-instrument and 8-instrument cassette sizes as preferred embodiments).

Hexagonally shaped apertures 106 further stabilize the individual-instrument grippers (preventing twisting);

Cassette lid 210 has plurality of hex-shaped apertures 106 to allow individual (non-paired) grippers 402 to house and anchor individual ‘odd shaped’ dental instruments 406 like pliers or syringes.

Cassette 102 can be loaded into an autoclave or ultrasonic machine either face up or face down while securely anchoring the dental instruments.

“Shovel plug and groove shaped clamp” topped 404 adjustable silicone grippers 404 (arranged as colored gripping sets) safely grip almost any dental instrument.

Unique color coordination of shaded-borders on hex tray and lid holes 106 (including optional color-coated anodized [optional] stainless steel [optional] itself on lid and tray) for easy insertion of same-colored gripping strips 402/204 to house elongated (and individual) instruments 406 (majority of border shading for standard perpendicular).

Traversing double-rail grippers 204, but diagonal and individual color coating for odd-shaped instruments is a [preferred] novel feature of the instant invention.

Optional “Grass-like” (“bristly”) Silicone [preferred] Clamping-Sheet Insert (not herein shown) provides additional stability and anchoring for instruments, while allowing the instant invention to maintain its “shovel and groove topped 404 gripping strips 402” to still function without impedance. The sheet acts as ballast, and as support, to further buttress the instruments against their rail 402 clamps 404, to prevent jostling.

What is claimed is:

1. A dental instrument housing apparatus, comprising:
a substantially porous dental cassette module configured to removably secure dental instruments, said cassette module comprising:
a substantially box-shaped main tray, comprising:
a rigid base, said base having two ends, said base in union with four walls disposed substantially perpendicular to the base;
at least one grooved rail, disposed longitudinally along at least one wall surface;
a drain system comprising a plurality of drainage apertures throughout said base and walls,
a removable lid cover with at least four side edges, comprising:
at least one ridge notch disposed longitudinally along at least one lid side edge, said ridge notch configured toengage with at least one of said grooved rails;
an irrigation system comprising a plurality of drainage apertures disposed throughout lid cover;
and,
at least one pliable gripping strip to hold dental instruments.

2. The apparatus of claim 1, wherein said gripping strip further comprises at least one plug fitting into at least one of said drainage apertures, removably engaged so as to secure and stabilize dental instruments, and wherein said gripping strip further comprises a shovel and groove top which clamps onto dental instruments.

3. The apparatus of claim 1, wherein the main tray further comprises at least one removable anti-slipfoot, said foot being plugged into at least one of said drainage apertures of said base.

4. The apparatus of claim 1, wherein the main tray further comprises at least two removable anti-slip feet, said feet being plugged into at least two of said drainage apertures of said base, wherein said feet are differently sized so as to elevate one end of said base higher than the other end of said base.

5. The apparatus of claim 1, wherein the main tray is substantially comprised of metal.

6. The apparatus of claim 1, wherein the main tray is substantially comprised of stainless steel.

7. The apparatus of claim 1, wherein the main tray base is substantially convex upward.

8. The apparatus of claim 1, wherein the main tray further comprises an adjustable wall comprised of at least one substantially pliable gripping strip, said strip comprising at least one plug fitting into at least one of said drainage apertures.

9. The apparatus of claim 1, wherein the lid cover further comprises at least one substantially pliable gripping strip, said strip comprising at least one plug fitting into at least one of said drainage apertures.

10. The apparatus of claim 1, wherein the main tray further comprises at least one substantially pliable removable gripping post, said gripping post comprising at least one plug fitting into at least one of said drainage apertures.

11. The apparatus of claim 1, wherein at least one aperture is substantially hexagonal.

12. The apparatus of claim 1, wherein said gripping strips comprise silicone.

13. A dental instrument housing apparatus, comprising:
a substantially porous dental cassette module configured to removably secure dental instruments, said cassette module comprising:
a substantially box-shaped main tray, comprising:
a rigid base, said base having two ends, said base in union with four walls disposed substantially perpendicular to the base;
at least one grooved rail, disposed longitudinally along at least one wall surface; and,
a drain system comprising a plurality of drainage apertures throughout said base and walls;
a removable lid cover with at least four side edges, comprising:
at least one ridge notch disposed longitudinally along at least one lid side edge, said ridge notch configured to engage with at least one of said grooved rails;
an irrigation system comprising a plurality of drainage apertures disposed throughout lid cover, wherein at least one drainage aperture is substantially hexagonal;
and further comprising,
at least one substantially pliable dental instrument gripping strip comprising at least one plug fitting into at least one drainage aperture;
at least one removable anti-slip foot, said foot being plugged into at least one of said drainage apertures of said base; and,
at least one substantially pliable removable gripping post, said gripping post comprising at least one plug fitting into at least one drainage aperture,
wherein,
said foot said gripping strip and said gripping post are comprised of the same pliable material and are interchangeably removably plugged into said drainage apertures to accommodate various-shaped dental instruments.

14. An apparatus, comprising:
a dental instrument cleaning cassette module configured to retain dental instruments to be disinfected based on irrigating the dental instruments with cleansing fluids flowing with a draining action through the cassette module, comprising:
a main tray, comprising:
a rigid container, open on one side, comprising a planar base surface section in mechanical union with walls disposed substantially perpendicular to the base surface;
a grooved gripping rail, disposed longitudinally along a wall end distal from the base surface and substantially parallel with the base surface;
a drain system comprising a plurality of hexagonal apertures disposed conformant with a main tray surface; and,
an adjustable gripping strip removably engaged with a main tray aperture;
and,
a lid adapted to removably cover the main tray open side, comprising:
a lid cover, comprising: a substantially planar lid cover section; and, a plurality of lid side edges in mechanical union with the lid cover and disposed substantially perpendicular to the lid cover;
a ridge notch disposed longitudinally along a lid side edge and configured to mechanically engage with a main tray grooved rail;
an irrigation system comprising a plurality of hexagonal apertures disposed substantially conformant with the lid cover; and,
an adjustable gripping strip removably engaged with a lid aperture.

15. The apparatus of claim 14, wherein the main tray further comprises a rubber foot mechanically engaged with the base surface section.

16. The apparatus of claim 14, wherein the cassette module further comprises a cant adapting package comprising a rubber foot mechanically engaged with a base surface section end and configured to adapt the main tray cant based on elevating the base surface section end above another base section end.

17. The apparatus of claim 14, wherein the main tray further comprises an adjustable wall disposed substantially perpendicular to the base surface section and configured to adjustably divide the container into segments having sizes determined as a function of the adjustable wall position.

18. The apparatus of claim 14, wherein the main tray further comprises an adjustable gripping rail.

19. The apparatus of claim 14, wherein the lid further comprises a corner tab.

20. The apparatus of claim 14, further comprising a removable bristly silicon sheet insertable between said rail and said lid.

21. An apparatus, comprising:
a dental instrument cleaning cassette module configured to retain dental instruments to be disinfected based on irrigating the dental instruments with cleansing fluids flowing with a draining action through the cassette module, comprising:
a main tray, comprising:
a rigid container, open on one side, comprising a planar base surface section in mechanical union with walls disposed substantially perpendicular to the base surface;
a grooved rail, disposed longitudinally along a wall end distal from the base surface and substantially parallel with the base surface; and,
a drain system comprising a plurality of hexagonal apertures disposed substantially conformant with a main tray surface;
an adjustable gripping strip removably engaged with a main tray aperture;
and,
a cant adapting package comprising a rubber foot mechanically engaged with a base surface section end and adapted to elevate the base surface section end above another base section end; and,
a lid adapted to removably cover the main tray open side, comprising:
a lid cover, comprising: a substantially planar lid cover section; and, a plurality of lid side edges in mechanical union with the lid cover and disposed substantially perpendicular to the lid cover;
a ridge notch disposed longitudinally along a lid side edge and configured to mechanically engage with a main tray grooved rail;
an irrigation system comprising a plurality of hexagonal apertures disposed substantially conformant with the lid cover; and,
an adjustable gripping strip removably engaged with a lid aperture.

22. The apparatus of claim 21, wherein the main tray further comprises a clamping-sheet insert.

23. The apparatus of claim 21, wherein the lid is treated with an antibiotic.

24. The apparatus of claim 21, wherein the gripping strip further comprises silicone treated with an antibiotic.

25. The apparatus of claim 21, wherein the lid further comprises a substantially convex surface.
